SEQUENCE LISTING (29 sequences)

SEQ ID NO: 1 - DNA, 20 bp, Artificial Sequence
  MAB2248 forward primers
gtgaccggtctcgatcagtt

SEQ ID NO: 2 - DNA, 20 bp, Artificial Sequence
  MAB2248 reverse primers
cattgtctggcgtcaatggc

SEQ ID NO: 3 - DNA, 30 bp, Artificial Sequence
  MAB2248 M. abscessus subsp. bolletii molecular beacon probe
ccgtgcctcgggaaccggatgactgcacgg

SEQ ID NO: 4 - DNA, 20 bp, Artificial Sequence
  MAB2830 forward primers
cctcatcgaggacggtcaga

SEQ ID NO: 5 - DNA, 20 bp, Artificial Sequence
  MAB2830 reverse primers
cacgaatccgggcagcaata

SEQ ID NO: 6 - DNA, 31 bp, Artificial Sequence
  MAB2830 M. abscessus subsp. abscessus molecular beacon probe
ccgtcgcgaggccggcatcggcgcacgacgg

SEQ ID NO: 7 - DNA, 15 bp, Artificial Sequence
  MAB2830 M. abscessus subsp. massiliense molecular beacon probe
ccgtcgcactacgag

SEQ ID NO: 8 - DNA, 20 bp, Artificial Sequence
  23S rrl forward primers
cggcgaaattgcactacgag

SEQ ID NO: 9 - DNA, 20 bp, Artificial Sequence
  23S rrl reverse primers
ccgaaccaaacgccaatacc

SEQ ID NO: 10 - DNA, 29 bp, Artificial Sequence
  23S rrl wild-type molecular beacon probe
cgctgccaggacgaaaagaccccgcagcg

SEQ ID NO: 11 - DNA, 20 bp, Artificial Sequence
  Erm(41) delta forward primers
ttcaggggagttcgttgtga

SEQ ID NO: 12 - DNA, 19 bp, Artificial Sequence
  Erm(41) delta reverse primers
aaagtgcttcgcggcaatg

SEQ ID NO: 13 - DNA, 24 bp, Artificial Sequence
  Erm(41) delta molecular beacon probe
cacggtggacgctccgggcccgtg

SEQ ID NO: 14 - DNA, 20 bp, Artificial Sequence
  Erm(41) T28C forward primers
gcatgccccgatatctttgg

SEQ ID NO: 15 - DNA, 20 bp, Artificial Sequence
  Erm(41) T28C reverse primers
atccacaacgaactcccctg

SEQ ID NO: 16 - DNA, 25 bp, Artificial Sequence
  Erm(41) T28C molecular beacon probe
cgctgacgccagcggggctgcagcg

SEQ ID NO: 17 - DNA, 34 bp, Artificial Sequence
  MAB2248 M. abscessus subsp. abscessus
ccgatcggagtcaaccggttcgcgggcatcgaac

SEQ ID NO: 18 - DNA, 34 bp, Artificial Sequence
  MAB2248 M. abscessus subsp. bolletii
ccgatcggagtcatccggttcccgagcatcgaac

SEQ ID NO: 19 - DNA, 34 bp, Artificial Sequence; identical to SEQ ID NO: 17
  MAB2248 M. abscessus subsp. massiliense

SEQ ID NO: 20 - DNA, 34 bp, Artificial Sequence
  MAB2830 M. abscessus subsp. abscessus
tcgatgcctgcgccgatgccggcctcgatgcggg

SEQ ID NO: 21 - DNA, 34 bp, Artificial Sequence
  MAB2830 M. abscessus subsp. bolletii
tcgatgcctgggccgataccggcctcgatgcggg

SEQ ID NO: 22 - DNA, 34 bp, Artificial Sequence
  MAB2830 M. abscessus subsp. massiliense
tcgatgcctggaccgataccggcttcgatgcggg

SEQ ID NO: 23 - DNA, 33 bp, Artificial Sequence
  23S rrl 2058-2059 wild-type
aaggtcccggggtcttttcgtcctgccgcgcgt

SEQ ID NO: 24 - DNA, 33 bp, Artificial Sequence
  23S rrl 2058-2059 AG mutant
aaggtcccggggtctcttcgtcctgccgcgcgt

SEQ ID NO: 25 - DNA, 33 bp, Artificial Sequence
  23S rrl 2058-2059 GA mutant
aaggtcccggggtcttctcgtcctgccgcgcgt

SEQ ID NO: 26 - DNA, 31 bp, Artificial Sequence
  Erm(41) T28C wild-type
gcggataccagccccactggcgtcgcgaccg

SEQ ID NO: 27 - DNA, 31 bp, Artificial Sequence
  Erm(41) T28C mutant
gcggataccagccccgctggcgtcgcgaccg

SEQ ID NO: 28 - DNA, 26 bp, Artificial Sequence
  Erm(41) delta wild-type
tgattccggcccgtagcgtccaatgg

SEQ ID NO: 29 - DNA, 26 bp, Artificial Sequence
  Erm(41) delta mutant
tgattccggcccggagcgtccagcgg

(19) United States
(12) Patent Application Publication
(10) Pub. No.: US 2024/0344151 A1
(43) Pub. Date: Oct. 17, 2024

(54) A Genotypic Assay To Subspeciate Mycobacterium Abscessus Complex Strains And Determine Macrolide Resistance

(71) Applicants: Hackensack Meridian Health, Inc., Edison, NJ (US); Rutgers, The State University of New Jersey, New Brunswick, NJ (US)

(72) Inventors: Barry N. Kreiswirth, New York, NY (US); Salvatore A. E. Marras, Roselle Park, NJ (US); Liang Chen, Livingston, NJ (US)

(73) Assignees: Hackensack Meridian Health, Inc., Edison, NJ (US); Rutgers, The State University of New Jersey, New Brunswick, NJ (US)

(21) Appl. No.: 18/289,859

(22) PCT Filed: May 9, 2022
(86) PCT No.: PCT/US2022/028321

§ 371 Date: Nov. 7, 2023

Related U.S. Application Data
(60) Provisional application No. 63/185,678, filed on May 7, 2021.

(51) Int. Cl. C12Q 1/689

(52) U.S. Cl. CPC C12Q 1/689; C12Q 2600/156

(57) ABSTRACT

A robust and rapid detection assay for the subspeciation of Mycobacterium abscessus is provided. The assay allows for three subspecies of M. abscessus to be detected: (1) M. abscessus subsp. abscessus, M. abscessus subsp. massiliense, and M. abscessus subsp. bolletii. Molecular beacon probes and primers were developed to detect the subspecies, and also determine which strains are susceptible macrolides.

The present invention relates to a genotyping assay that can subspeciate Mycobacterium abscessus into three subspecies: abscessus, massiliense, and. bolletii. In particular, the genotyping assay includes a real-time multiplex assay using molecular beacon probes to establish a robust, rapid and highly accurate method to distinguish the subspecies of Mycobacterium abscessus and determine which strains may be susceptible to macrolides.

### BACKGROUND OF THE INVENTION

Mycobacterium abscessus is a rapidly growing nontuberculous mycobacterial species that comprises the following subspecies: abscessus, massiliense, and bolletii. M. abscessus has been associated with skin and soft-tissue infections resulting from contaminated equipment and hospital water supplies, but more recently, it has emerged as a life-threatening chronic pulmonary pathogen in both immunocompetent and immunocompromised patients. For example, M. abscessus has become a significant cause of chronic lung infections in persons with cystic fibrosis (CF). The treatment is confounded by their intrinsic resistance to antibiotics, including anti-tuberculosis agents. The inability to successfully treat M. abscessus infections with macrolides, clarithromycin or azithromycin, as a result of the erm(41) gene and mutations in the 23S rrl gene, has dramatically impacted patient outcomes, where cure rates drop to 25-40% against clarithromycin resistant M. abscessus.

As a result of the increased number of high-risk immunocompromised patient populations, including bone marrow transplant patients and those receiving solid organs, and those with underlying lung disease, such as cystic fibrosis patients, there is an increase in M. abscessus chronic infections and a dire clinical need to improve patient treatment. Macrolide antibiotics remain one of the most effective classes of antibiotics against susceptible M. abscessus strains; however, standard microbiology laboratories typically have limited diagnostic tools for the subspeciation of M. abscessus, and the testing for macrolide resistance is often not done. Further, M. abscessus is a slow growing environmental bacteria, where culturing pathogens from samples could take between 3 to 4 weeks. If susceptibility testing were further performed, the results would take an additional month from culturing. Thus, this creates a two month lapse in providing appropriate antibacterial treatment.

As is understood in the art, subspeciation of M. abscessus and genotypic resistance are linked to macrolides, including the important observation that nearly all M. abscessus subspecies massiliense strains have two deletions (one of 2 bp and the other of 274 bp, bases 64 to 65 and 159 to 432) in the erm(41) gene that leads to susceptibility to clarithromycin. Similarly, there are clinical isolates of M. abscessus subspecies abscessus with a single base change from T to C in codon 28 in the erm(41) gene that correlates with clarithromycin susceptibility.

The diagnostic challenge of developing a rapid and simple assay to both subspeciate M. abscessus and to genotype macrolide resistance revolves around the requirement to sequence the various subspecies and drug susceptibility gene targets. A number of examples, including genetic changes in rpoB, hsp65, and secA1 have been used to subspeciate M. abscessus with relatively high accuracy, but in each case, the assay requires PCR amplification followed by DNA sequencing. The same issue applies to define the resistance mutations in the 23S rrl gene and the erm(41) genetic alterations that are associated with macrolide susceptibility in M. abscessus subspecies abscessus and M. abscessus subspecies massiliense. PCR approaches that require gel electrophoresis have been developed; but these research techniques are labor intensive, are subjective in interpreting the results, and are not amenable to the routine clinical microbiology laboratory.

Additionally, multiplex PCR based target next generation sequencing and Cas12a/sgRNA-based nucleic acid detection platforms were recently developed for M. abscessus subspeciation. Commercial nucleic acid amplification tests, such as the line probe GenoType NTM-DR assay, which distinguish the M. abscessus subspecies and macrolide resistance, are also available. However, all these approaches also involve post PCR procedures using isolated cultures, which require longer turnaround times. Due to the high level of genetic relatedness, it is a challenge to differentiate strains from M. abscessus based on a single gene sequence

Accordingly, there exists a need to improve the diagnosis of the subspecies of M. abscessus and to genotype the macrolide resistance to determine drug susceptibility gene targets using a single, rapid assay.

### BRIEF SUMMARY OF THE INVENTION

An assay for detecting different species of the Mycobacterium abscessus and detecting a mutation including a forward primer for MAB2248 represented by SEQ ID NO. 1; a reverse primer for MAB2248 represented by SEQ ID NO. 2; a molecular beacon probe for detecting M. abscessus subsp. bolletii reperesented by SEQ ID NO. 3; a forward primer for MAB2830 represented by SEQ ID NO. 4; a reverse primer for MAB2830 represented by SEQ ID NO. 5. The assay also includes a molecular beacon probe for detecting M. abscessus subsp. abscessus represented by SEQ ID NO. 6; a molecular beacon probe for detecting M. abscessus subsp. massiliense represented by SEQ ID NO. 7; a forward primer for 23S rrl represented by SEQ ID NO. 8; a reverse primer for 23S rrl represented by SEQ ID NO. 9. The assay further includes a molecular beacon probe for detecting the wild type of 23S rrl represented by SEQ ID NO. 10; a forward primer for erm(41) A represented by SEQ ID NO. 11; a reverse primer for erm(41) A represented by SEQ ID NO. 12; a molecular beacon probe for erm(41) A represented by SEQ ID NO. 13; a forward primer for erm(41) T28C represented by SEQ ID NO. 14; a reverse primer for erm(41) T28C represented by SEQ ID NO. 15; and a molecular beacon probe for detecting erm(41) T28C represented by SEQ ID NO. 16.

A method for detecting different species of the Mycobacterium abscessus and detecting a mutation, including preparing an assay, including a molecular beacon probe in the assay to determine a species of the Mycobacterium abscessus, including a primer in the assay to detect a mutation, performing a real time-polymerase chain reaction (RT-PCR) on the assay, and analyzing the assay for the species of the Mycobacterium abscessus and the mutation.

### BRIEF DESCRIPTION OF THE DRAWINGS

FIG. 1 illustrates the denaturation profile analysis of the six molecular beacon probes used in the assay of the present application.

FIG. 2 illustrates the two-tube real-time PCR assay results of Assays I and II.

### DETAILED DESCRIPTION

The term “assay” refers to a standardized procedure to detect the presence or absence of a particular nucleic acid of interest and is a genotyping assay. The term “species” refers to subspecies of M. abscessus.

The assay of the present application is a real-time transcription polymerase chain reaction (RT-PCR) assay. The assay is a novel genotyping assay that may subspeciate M. abscessus into three species. The three species may include abscessus, massiliense and bolletti. The assay may further determine the presence of specific mutations to predict the strains susceptibility to macrolide treatment. The assay of the present application may provide results within about 2 to 3 hours. Accordingly, the assay of the present application is ideal to serve as a rapid and robust tool to facilitate detection of a subspecies of M. abscessus and identifying mutations to predict susceptibility to macrolide treatment.

In one embodiment, the assay may be a two-tube, three-fluoresence-tagged multiplex genotyping assay including molecular beacon probes. In another embodiment, the assay may be an eight-tube, three-fluoresence-tagged multiplex genotyping assay including molecular beacon probes. The molecular beacon probes are designed to identify sequence targets that are unique to two genes, MAB2248 and MAB2830. In particular, molecular beacon probes may be phylogenetically mined. The identified subspecies-specific regions provide 100% sensitivity and specificity, both in silico and in experimental assays.

For the identification and development of subspecies-specific sequence targets that are amenable for RT-PCR and molecular beacon probe detection, 33 completely sequenced M. abscessus genomes from the National Center for Biotechnology Information (NCBI) were integrated including: 12 M. abscessus subsp. abscessus, 19 M. abscessus subsp. massiliense and 2 M. abscessus subsp. bolletii. The genomes were aligned by Parsnp from Harvest suite, and subspecies specific single nucleotide polymorphisms (SNPs) were extracted. A core genome analysis was conducted using Roary, as understood by one of skill in the art, to identify conserved genes across different M. abscessus sub-species. Core SNP numbers within a 20-nt sliding window across M. abscessus core genes were calculated using BEDtools, as understood in by one of skill in the art, to identify putative regions for molecular beacon probe design.

To facilitate the ability to select alternate targets for M. abscessus detection and subspeciation, 1,930 M. abscessus genomes were analyzed. From this analysis, two targets for SNP based real-time PCR design. Blastn was used to examine the in silico sensitivity and specificity of putative molecular beacon probes and their neighboring sequences against 1,930 Mycobacterium abscessus complex (MABC) draft genomes in the NCBI assembly database and among in-house sequenced genomes. In addition, the specificity of the molecular beacons and primers were also examined against 476 representative or completely sequenced genomes of nontuberculous mycobacterial species in the NCBI Refseq database, including genomes from 119 unique mycobacterial species or subspecies.

Acquired macrolide resistance in M. abscessus is largely associated with the point mutations A2058 and A2059 in the rrl gene encoding the peptidyltransferase domain of the 23S rRNA, and inducible macrolide resistance is linked to the ribosomal methylase gene erm(41). The erm(41) gene with a T-to-C polymorphism at nt 28 is associated with macrolide susceptibilty. Currently, most clinical isolates of M. abscessus subsp. abscessus and M. abscessus subsp. bolletii express inducible macrolide resistance but in contrast, M. abscessus subsp. massiliense is nearly always associated with clarithromycin susceptibility due to two deletions, bases 64 to 65 and 159 to 432, within erm(41). This association correlates with a favorable clinical outcome with macrolide treatment. Three sets of primer pairs and molecular beacon probes to detect the rrl macrolide susceptible (wild-type) gene, the erm(41) deletion and the erm(41) T28C mutation in order to specifically and rapidly genotype macrolide resistance in different M. abscessus sub-species. A region in gene MAB2248 and two regions in MAB2830 were used to design three primer pairs and molecular beacon probes to specifically identify M. abscessus subsp. abscessus, M. abscessus subsp. bolletii, and M. abscessus subsp. massiliense, respectively.

The PCR primers for use in the assay were designed utilizing Primer-BLAST. The molecular beacon probes were designed to identify gene or species-specific single nucleotide polymorphisms using guidelines described by Vet and Marras in “Design and optimization of molecular beacon real-time polymerase chain reaction assays” Methods Mol. Biol 288:273-290. The PCR primers and molecular beacon probes were evaluated with IDT's OligoAnlyzer Tool software for compatibility in multiplex PCR assays. The sequences of the PCR primers and molecular beacon probes of the present application are listed in Table 1.

TABLE 1
Molecular beacon probe and primer sequences

| Oligonucleotide^{A} | SEQ ID NO. | Sequence (5′ - 3′)^{B} |
|---|---|---|
| MAB2248 for | SEQ ID NO. 1 | GTGACCGGTCTCGATCAGTT |
| MAB2248 rev | SEQ ID NO. 2 | CATTGTCTGGCGTCAATGGC |
| MAB2248 bol MB | SEQ ID NO. 3 | CFR610 - CCGTGCCTCGG |
| | | GAACCGGATGACTGCACG |
| | | G - BHQ-2 |
| MAB2830 for | SEQ ID NO. 4 | CCTCATCGAGGACGGTCAGA |
| MAB2830 rev | SEQ ID NO. 5 | CACGAATCCGGGCAGCAATA |
| MAB2830 abs MB | SEQ ID NO. 6 | FAM - CCGTCGCGAGGCCG |
| | | GCATCGGCGCACGACGG - |
| | | BHQ-1 |
| MAB2830 mas MB | SEQ ID NO. 7 | Q670 - CCGTCGCACTACG |
| | | AG |
| 23S rrl for | SEQ ID NO. 8 | CGGCGAAATTGCACTACGAG |
| 23S rrl rev | SEQ ID NO. 9 | CCGAACCAAACGCCAATACC |
| 23S rrl WT MB | SEQ ID NO. 10 | FAM - CGCTGCCAGGACGA |
| | | AAAGACCCCGCAGCG - |
| | | BHQ-1 |
| erm(41) A for | SEQ ID NO. 11 | TTCAGGGGAGTTCGTTGTGA |
| erm(41) A rev | SEQ ID NO. 12 | AAAGTGCTTCGCGGCAATG |
| erm(41) A MB | SEQ ID NO. 13 | CFR610 - CACGGTGGACG |
| | | CTCCGGGCCCGTG - |
| | | BHQ-2 |
| erm(41) T28C for | SEQ ID NO. 14 | GCATGCCCCGATATCTTTGG |
| erm(41) T28C rev | SEQ ID NO. 15 | ATCCACAACGAACTCCCCTG |
| erm(41) T28C MB | SEQ ID NO. 16 | Q670 - CGCTGACGCCAGC |
| | | GGGGCTGCAGCG - |
| | | BHQ-2 |

^{A}forward (for) and reverse (rev) primers, and molecular beacon probe (MB) sequences for each of the target genes. bol = M. abscessus subsp. bolletii, abs = M. abscessus subsp. abscessus, mas = M. abscessus subsp. massiliense.
^{B}FAM = fluorescein, CFR610 = Cal Fluor Red 610, Q670 = Quasar 670, BHQ-1 = Blackhole Quencher 1, BHQ-2 = Blackhole Quencher 2, underlined nucleotides represent arm sequences of the molecular beacon probe.

Two RT-PCR assays were performed for each sample of a culture or isolated bacteria of interest. One assay, Assay I, contained PCR primers and molecular beacon probes to determine the presence of M. abscessus subsp. bolletii, the wild-type gene for 23S rrl 2058-2059, and the erm(41) T28C mutation. The second assay, Assay II, contained PCR primers and molecular beacon probes to determine the presence of either M. abscessus subsp. abscessus or M. abscessus subsp. massiliense, and the erm(41) deletion. Each RT-PCR assay was carried out in 20-μL volumes that contained 1× Platinum Hot Start PCR Master Mix (Thermo Fisher Scientific, Waltham, MA). For Assay I, 250 nM MAB2248 for, 250 nM MAB2248 rev, 500 nM MAB2248 bol MB, 250 nM 23S rrl for, 250 nM 23S rrl rev, 500 nM 23S rrl MB, 100 nM erm(41) T28C for, 1,000 nM erm(41) T28C rev and 500 nM erm(41) T28C MB was added. For Assay II, 250 nM MAB2830 for, 250 nM MAB2830 rev, 500 nM MAB2830 abs MB, 250 nM MAB2830 mas MB, 250 nM erm(41) A for, 250 nM erm(41) A rev and 500 nM erm(41) Δ MB was added. Each assay was initiated with a 5 μl DNA template. The PCR assays were performed in 200-μL white polypropylene PCR tubes in a CFX96 Touch Real-Time PCR Detection System. The reaction mixtures were incubated for 2 minutes at 95° C. to activate the DNA polymerase, followed by 40 thermal cycles that consisted of 95° C. denaturation for 15 seconds and 60° C. annealing and chain elongation for 60 seconds. Molecular beacon fluorescence intensity was monitored during the 60° C. annealing and chain elongation stage of each thermal cycle.

The molecular epidemiology of the three subspecies varies significantly, and infections caused by M. abscessus subsp. bolletii are rarer in comparison to the other two subspecies. However, clinical disease and outbreaks caused by M. abscessus subsp. bolletii have been documented. In contrast to M. abscessus subsp. bolletii, M. abscessus subsp. abscessus and M. abscessus subsp. massiliense postsurgical wound infections and respiratory disease are frequently reported globally and differences in clinical outcomes commonly correlate to the strain's susceptibility to macrolides. Infections due to M. abscessus subsp. massiliense respond more favorably to macrolide therapy. However, as shown in the present application, not all M. abscessus subsp. massiliense harbor the truncated erm(41) and M. abscessus subsp. massiliense isolates with a wild-type erm(41) have been described. The erm(41) C28 sequevar M. abscessus subsp. abscessus are associated with significantly higher culture conversion rates with treatment, since these isolates do not exhibit inducible resistance to macrolides. Finally, M. abscessus subsp. massiliense with the truncated erm(41) gene have been identified with mutations in the rrl 2058 and 2059 sites, making them fully resistant to macrolides. Collectively, subspeciation and genotyping macrolide susceptibility are necessary to guide precise treatment for clinical M. abscessus infections. The assay of the present application was designed to detect the most common mutations associated with macrolide resistance in MABC, but is not limited, for example, a rare resistance mechanism could be targeted and added to the assay if they become more prevalent among clinical isolates.

The assay of the present application has strong predictive ability to determine whether to use a macrolide in the patient's treatment regimen. Compared to the 3-14 days needed for clarithromycin phenotypic susceptibility testing, the real-time PCR method of the present application can be completed in 2-3 hours, providing timely and definitive laboratory results. From the studies described below, it is also suggested that the erm(41) gene truncation is not a reliable marker to subspeciate M. abscessus subsp. massiliense. Importantly, the assay showed 100% specificity and sensitivity among 115 tested M. abscessus strains from multiple healthcare facilities in the US, further supporting its clinical application. The ability for healthcare facilities to extend their diagnostic capability in the treatment of M. abscessus infections would dramatically improve patient care by providing appropriate antibiotic treatment and establish epidemiological markers to monitor and track M. abscessus infections.

The assay of the present application will be described through the following tests and experiments.

### Clinical Isolates and Clarithromycin Testing

A collection of 115 M. abscessus clinical isolates, including 100 recovered from patients with cystic fibrosis and catalogued and archived at National Jewish Health, were utilized. The strains included 69 M. abscessus subsp. abscessus, 38 M. abscessus subsp. massiliense and 10 M. abscessus subsp. bolletii. All strains were analyzed by whole genome sequencing (bioproject accession no. PRJNA319839 and PRJNA549322). A collection of 22 strains from 20 different nontuberculous Mycobacterium species, including M. aurum, M. avium, M. gallinarum, M. gastri, M. gordonae, M. haemophilum, M. intracellulare, M. marinum, M. scrofulaceum, M. simiae, M. szulgai, M. terrae, M. ulcerans, M. chelonae, M. fortuitum, M. kansasii, M. phlei, M. simiae, plus M. tuberculosis and M. microti, archived at the Center for Discovery and Innovation were used to evaluate the specificity of the assay.

A panel of six test strains, that include all three subspecies and genotypic diversity associated with clarithromycin resistance, were analyzed for their response to clarithromycin. Susceptibility and resistance were assessed according to the CLSI recommendations. Constitutive clarithromycin resistance was defined by a MIC≥8 μg/ml at day 5. Inducible resistance was defined by an increase in clarithromycin MIC from≤2 μg/ml at day 5 to ≥8 g/ml at day 14.

### Target Selection, In-Silico Sensitivity and Specificity

Genomic analysis of 33 whole genome sequenced (WGS) M. abscessus genomes identified candidate genetic regions that were amenable to subspeciate M. abscessus isolated using the molecular beacon probes of the present application. Two gene targets, MAB2248 and MAB2830, were selected based on their conservation and specificity to differentiate the three subspecies. MAB2248 encodes a putative peptide synthetase MbtE protein and MAB2830 is the dihydroorotase PyrC gene. An 18-bp sequence within the MAB2248 gene, specific to M. abscessus subsp. bolletii, was targeted for the molecular beacon probe to distinguish this subspecies. The MAB2248 beacon differs from the sequences of M. abscessus subsp. abscessus or M. abscessus subsp. massiliense by three SNPs ( 3/18, 16.7% variation), providing the specificity to detect M. abscessus subsp. bolletii in an allelic specific RT-PCR assay. MAB2830 homologous sequences in M. abscessus subsp. abscessus and M. abscessus subsp. massiliense were used to design the two subspecies-specific molecular beacons, and the target regions can be amplified by the same PCR-primer pairs in the two subspecies. An 18 bp MAB2830 sequence, specific for M. abscessus subsp. abscessus, was used as a molecular beacon probe and this sequence is distinguishable from M. abscessus subsp. massiliense and M. abscessus subsp. bolletii by 4-5 SNPs (22.2-27.8% variations), respectively. Similarly, a 19 bp MAB2830 homolog sequence, specific to M. abscessus subsp. massiliense, was targeted for the design of the molecular beacon probe and differed from the MAB2830 sequences in M. abscessus subsp. abscessus and M. abscessus subsp. bolletii by 5 and 3 SNPs (16.7-27.8% variations), respectively.

The in-silico sensitivity and specificity of the three designed molecular beacon probes and primers were evaluated against 1,930 M. abscessus genomes from NCBI and from the inventor's own collection. The 1,930 genomes were assigned to three subspecies and they include 1,192 M. abscessus subsp. abscessus, 609 M. abscessus subsp. massiliense and 129 M. abscessus subsp. bolletii. MAB2248 and MAB2830 sequences were found in all 1,930 MABC genomes. The in silico sensitivities were 100%, 99.41% and 99.67% for M. abscessus subsp. bolletii, M. abscessus subsp. massiliense and M. abscessus subsp. abscessus specific molecular beacons, and the specificities were 99.72%, 100% and 99.92% for the three subspecies, respectively, as is seen in Table 2.

TABLE 2
In silico sensitivity and specificity of subspeciation PCR probes

| MAB2248 | WGS | | MAB2830 | WGS | | MAB2830 | WGS | |
|---|---|---|---|---|---|---|---|---|
| bol MB | + | − | abs MB | + | − | mas MB | + | − |
| PCR+ | 129 | 5 | PCR+ | 1185 | 0 | PCR+ | 607 | 1 |
| PCR− | 0 | 1796 | PCR− | 7 | 738 | PCR− | 2 | 1320 |
| Sensitivity | 100 | | Sensitivity | 99.41 | | Sensitivity | 99.67 | |
| Specificity | 99.72 | | Specificity | 100 | | Specificity | 99.92 | |
| PPV | 96.27 | | PPV | 100 | | PPV | 99.84 | |
| NPV | 100 | | NPV | 99.06 | | NPV | 99.85 | |

PPV, positive predictive value; NPV, negative predictive value.

To confirm that the assay may be used to diagnose primary patient specimens, the in-silico specificity of the three subspecies molecular beacon probes and two primer pairs against 476 representative or completely sequenced Mycobacterium genomes deposited in the NCBI Refseq database. The results showed the primers and probes are highly specific to M. abscessus strains, showing no or low sequence identities to other Mycobacterium species. Among the most clinically relevant nontuberculous Mycobacterium species described above, the MAB2248 primer and probe sequences were absent in all but the five species that belong to the M. chelonae complex (M. chelonae, M. saopaulense, M. immunogenum, M. salmoniphilum and M. franklinii) and when present, the molecular beacon target region only showed 61-82% (4-7 mismatches) similarities. Similarly, the MAB2830 target was absent in most species and revealed 70-80% (4-6 mismatches) nucleotide identity against only genomes from M. chelonae, M. saopaulense, M. immunogenum, M. salmoniphilum and M. franklinii. In addition, the two gene targets were absent in other CF and lung associated pathogens, including Staphylococcus aureus, Haemophilus influenzae, Pseudomonas aeruginosa, Burkholderia cepacia complex, and Stenotrophomonas maltophilia. The in-silico specificity examination of the primers and probes for the erm(41) T28C and the deletion were specific for the M. abscessus genomes, and there are no sequence matches against other queried genomes. The 23S rrl primers and molecular beacon were designed to target the wild-type alleles (A2058 and A2059), and this region showed 100% identity to other species of M. chelonae, M. saopaulense, M. immunogenum, M. salmoniphilum and M. franklinii.

### Denaturation Profile Analysis

The molecular beacon probes described above for use in the assay, were characterized in a denaturation profile analysis to determine that they only elicit a fluorescence signal if their intended perfect matched target is present at the annealing temperature of the PCR and remain non-fluorescent if a non-intended mismatched target is present at the annealing temperature of the PCR. For each of the three molecular beacon probes that distinguish the M. abscessus subspecies present in a sample, three oligonucleotide targets were designed, as can be seen in Table 3: one oligonucleotide is identical to the target region of the probe to M. abscessus subsp. abscessus; one to the target region to the M. abscessus subsp. massiliense probe; and one to the target region to the probe for M. abscessus subsp. bolletii. Each analysis also included a control in which no oligonucleotide target was added, and this control showed at which temperature the molecular beacon probe stem opens, resulting in an increase in the background fluorescence of the probe.

TABLE 3
Denaturation profile analysis
target oligonucleotides

| Oligo- | |
|---|---|
| nucleotide^{A} | |
| SEQ ID NO. | Sequence (5′-3′) |
| MAB2248 abs | CCGATCGGAGTCAACCGGTTCGCGGGCATCGAAC |
| SEQ ID NO. 17 | |
| MAB2248 bol | CCGATCGGAGTCATCCGGTTCCCGAGCATCGAAC |
| SEQ ID NO. 18 | |
| MAB2248 mas | CCGATCGGAGTCAACCGGTTCGCGGGCATCGAAC |
| SEQ ID NO. 19 | |
| MAB2830 abs | TCGATGCCTGCGCCGATGCCGGCCTCGATGCGGG |
| SEQ ID NO. 20 | |
| MAB2830 bol | TCGATGCCTGGGCCGATACCGGCCTCGATGCGGG |
| SEQ ID NO. 21 | |
| MAB2830 mas | TCGATGCCTGGACCGATACCGGCTTCGATGCGGG |
| SEQ ID NO. 22 | |
| 23S rrl | AAGGTCCCGGGGTCTTTTCGTCCTGCCGCGCGT |
| 2058-2059 wt | |
| SEQ ID NO. 23 | |
| 23S rrl | AAGGTCCCGGGGTCTCTTCGTCCTGCCGCGCGT |
| 2058-2059 AG | |
| mut | |
| SEQ ID NO. 24 | |
| 23S rrl | AAGGTCCCGGGGTCTTCTCGTCCTGCCGCGCGT |
| 2058-2059 | |
| GA mut | |
| SEQ ID NO. 25 | |
| erm(41)T28C | GCGGATACCAGCCCCACTGGCGTCGCGACCG |
| wt | |
| SEQ ID NO. 26 | |
| erm(41) T28C | GCGGATACCAGCCCCGCTGGCGTCGCGACCG |
| mt | |
| SEQ ID NO. 27 | |
| erm(41) A wt | TGATTCCGGCCCGTAGCGTCCAATGG |
| SEQ ID NO. 28 | |
| erm(41) A mut | TGATTCCGGCCCGGAGCGTCCAGCGG |
| SEQ ID NO. 29 | |

^{A}abs = M. abscessus subsp. abscessus, bol = M. abscessus subsp. bolletii, mas = M. abscessus subsp. massiliense, wt = wild-type, mut = mutant.

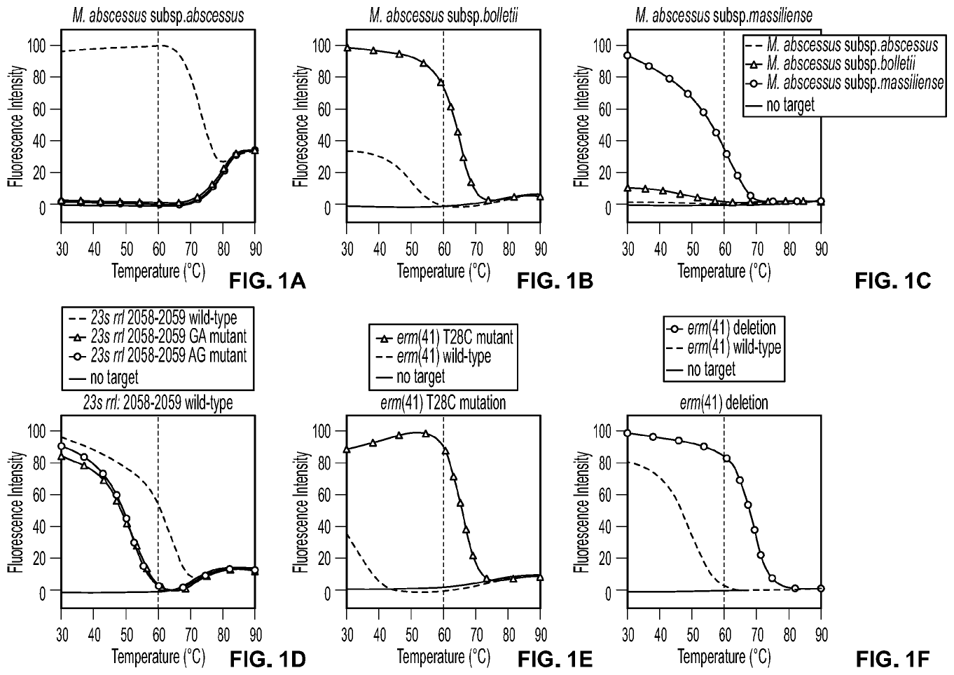

In FIG. 1, the top row illustrates the denaturation profiles of the three subspecies molecular beacon probes. The plots show that at the annealing temperature of the PCR assay (60° C., and represented by a dashed line), a fluorescent signal only arose from the subspecies-specific molecular beacon probe and its intended perfect matched target oligonucleotide. At this temperature, the probes did not elect a fluorescence signal from the other two, mismatched, gene targets. Furthermore, the stem of the molecular beacon probes was closed at this temperature and there was no increased background fluorescence from the probes. Similar denaturation profile analyses were carried out for the three molecular beacon probes designed to identify the wild type rrl gene, the erm(41) T28C mutation, and the erm(41) deletion. For these probes, oligonucleotide targets were designed as a perfect matched for the intended target and with mismatches compared to non-intended targets. In FIG. 1, the bottom row illustrates the denaturation profiles of these three molecular beacon probes. For example, the left plot shows the denaturation profile for the wild type rrl gene molecular beacon probe in the presence of its intended, wild-type, target (green curve) and in the presence of the two possible mismatched targets, a GA mutant (red curve) or AG mutant (blue curve), and in absence of any target (black curve) as a control to determine the stability of the stem hybrid of the probe. The plot shows that at the 60° C. annealing temperature, only a fluorescence signal from the probe arose when its intended perfect matched, wild-type, target was present. At this temperature, the molecular beacon probe did not show any fluorescence in the presence of the two non-intended mutant targets, indicating the perfect discrimination of this molecular beacon probe between the wild-type and mutant targets. This was also the case for the other molecular beacon probes in this assay.

### Assay Analysis

The real-time PCR results were analyzed. From this analysis, the information for both multiplex assays were combined to predict the M. abscessus subspecies and to determine whether the strain is macrolide resistant or susceptible. The assay was designed with the understanding that all isolates have the erm(41) gene and its wild type structure correlates with inducible macrolide resistance. Therefore, those strains that do not have the T28C mutation or the 274 bp deletion are predictively resistant. Similarly, the wild type 23S rrl gene is associated with macrolide susceptibility and rare strains have a mutation that correlate with constitutive macrolide resistance. In the current assay, the absence of the rrl positive molecular beacon predicts resistance.

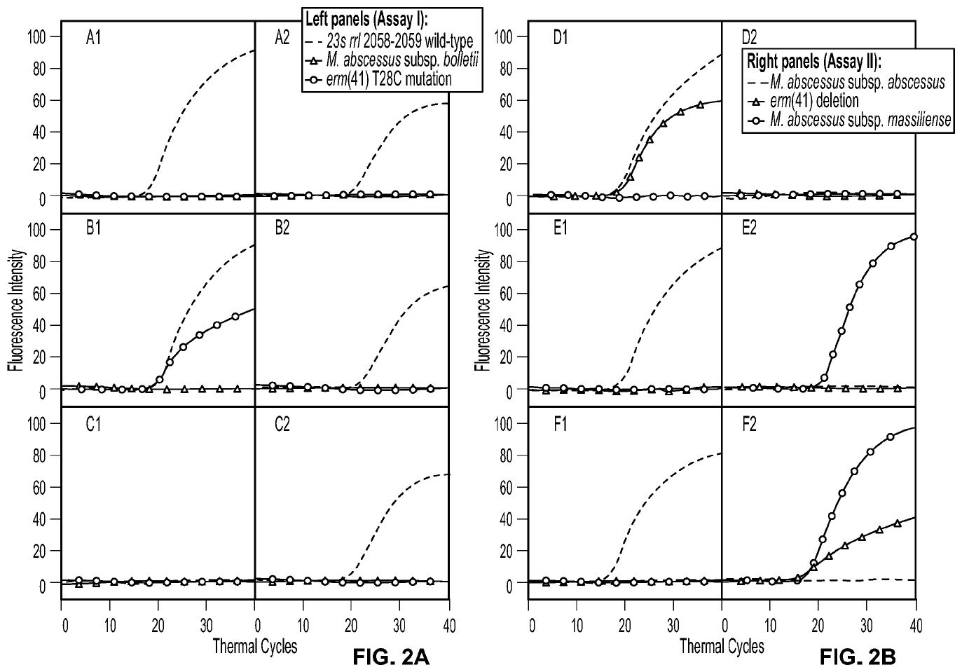

The multiplex real-time PCR results of assays I and II are shown in FIG. 2. The genotype of 6 representative clinical strains are described in Table 4.

TABLE 4
Interpretation of real-time PCR results for six MABC isolates

| | Real-time PCR assay I* | | | Real-time PCR assay II* | | | Results interpretation | |
|---|---|---|---|---|---|---|---|---|
| | MAB2248 | 23S rrl WT | erm(41) | MAB2830 | MAB2830 | erm(41) Δ | | Macrolide |
| Strains | bol (MB) | (MB) | T28C (MB) | abs (MB) | mas (MB) | (MB) | Subspecies | Phenotype |
| A | − | + | − | + | − | − | abscessus | Inducible |
| | | | | | | | | Resistance |
| B | − | + | + | + | − | − | abscessus | Susceptible |
| C | − | − | − | + | − | − | abscessus | Constitutive |
| | | | | | | | | Resistance |
| D | + | + | − | − | − | − | bolletii | Inducible |
| | | | | | | | | Resistance |
| E | − | + | − | − | + | − | massiliense | Inducible |
| | | | | | | | | Resistance |
| F | + | + | + | + | + | + | massiliense | Susceptible |

*“+”, positive amplification for the molecular beacon; “−”, negative amplification for the molecular beacon

For each strain, the real-time PCR profiles of Assay I is shown in the left panel and the profiles of Assay II are shown in the right panel in FIG. 2. The results for strain A revealed the presence of the wild type rrl gene and the identification of M. abscessus subsp. abscessus. The interpretation is that this strain has the wild type erm(41), wild type rrl gene and consequently, it had inducible macrolide resistance. The results for strain B showed the wild type rrl gene, but also the presence of the erm(41) T28C mutation, indicating that this strain was susceptible to macrolides, and the positive FAM molecular beacon profile in the right panel indicates it was an M. abscessus subsp. abscessus. The absence of a positive molecular beacon profile for strain C in the left panel indicated that it had the rrl gene mutation, predictive of constitutive macrolide resistance, and the positive FAM molecular beacon profile in the right panel indicates it was an M. abscessus subsp. abscessus. The results for strain D revealed the wild type rrl gene and a positive CFR610 molecular beacon fluorescence in the left panel, predictive of M. abscessus subsp. bolletii. The absence of any molecular beacon profile in the right panel confirmed the subspeciation and the overall interpretation that this was an M. abscessus subsp. bolletii with inducible resistance to macrolides as a result of having the wild type erm(41) gene. The results for strains E and F revealed the wild type rrl gene and a positive Q670 fluorescence in the right panel, predictive of M. abscessus subsp. massiliense. For strain E, the erm(41) showed a wild type genotype, indicating this strain had inducible resistance to macrolides, whereas strain F had the definitive deletion that was predictive of a macrolide susceptible strain.

### Technical Sensitivity and Specificity

The sensitivity of the assay was determined by initiating PCR assays with different quantities of DNA obtained from strains B, D and F. These strains were chosen, because together they contain target regions for all six molecular beacon probes utilized in this assay. The amount of DNA added as a template to each of the reactions was calculated to be equivalent to 1,000, 100, 10 and 1 copies of genomic DNA. The results showed that Assays I and II for each strain was able to detect and identify 1 or 10 copies of genomic DNA.

We tested the specificity of the assay using DNAs from 19 different Mycobacterium species. No positive amplifications were observed for MAB2248 and MAB2830 molecular beacon probes, which was consistent with the above in silico data mining results. The bioinformatic and experimental approaches confirm that the assay is highly specific for the detection of M. abscessus. In addition, none of the 19 sample panel was positive for the erm(41) T28C mutation or erm(41) 274-bp deletion. For the 23S rrl molecular beacon, only DNA from M. chelonae gave a positive amplification with Ct value<30 cycles, while other species only showed very late amplification signals (Ct>33). It is significant to note that the 23S rrl primer and molecular beacon regions are 100% identical between M. abscessus and M. chelonae and in agreement with the in silico data, the rrl RT-PCR result was positive.

### Clinical Isolate Testing

The assay was used to evaluate a large collection of genetically characterized, clinical M. abscessus isolates from National Jewish Health, representing NTM isolates from CF centers across the United States, and from selected strains archived at the Center for Discovery and Innovation. Blinded DNA samples were assayed and compared to the subspeciation and the macrolide genotype of each strain as determined by whole genome sequencing. A summary of each of the molecular beacon results are shown in Table 5 for a total of 115 strains that were assayed. The results showed 100% specificity and sensitivity in determining the sub-species and the macrolide genotype and the individual results are shown in Table 6.

TABLE 5
Performance of the real-time PCR in 115 clinical MABC isolates in comparison to the whole genomic sequencing result

| | | Whole genome sequencing analysis results | | | | | | | | | | | |
|---|---|---|---|---|---|---|---|---|---|---|---|---|---|
| | | | | | | | | 23s rrl: 2058-2059 | | erm(41) | | erm(41) T28C | |
| | | abs | | mas | | bol | | mutants* | | truncation | | mutation | |
| | | yes | no | yes | no | yes | no | yes | no | yes | no | yes | no |
| MB-real-time PCR results | MAB2830 abs | 69 | 0 | | | | | | | | | | |
| | MB pos | | | | | | | | | | | | |
| | MAB2830 abs | 0 | 46 | | | | | | | | | | |
| | MB neg | | | | | | | | | | | | |
| | MAB2830 mas | | | 36 | 0 | | | | | | | | |
| | MB pos | | | | | | | | | | | | |
| | MAB2830 mas | | | 0 | 79 | | | | | | | | |
| | MB neg | | | | | | | | | | | | |
| | MAB2248 bol | | | | | 10 | 0 | | | | | | |
| | MB pos | | | | | | | | | | | | |
| | MAB2248 bol | | | | | 0 | 105 | | | | | | |
| | MB neg | | | | | | | | | | | | |
| | 23S rrl | | | | | | | 4 | 0 | | | | |
| | MB pos | | | | | | | | | | | | |
| | 23S rrl | | | | | | | 0 | 111 | | | | |
| | MB neg | | | | | | | | | | | | |
| | erm(41) Δ | | | | | | | | | 35 | 0 | | |
| | MB pos | | | | | | | | | | | | |
| | erm(41) Δ | | | | | | | | | 0 | 80 | | |
| | MB neg | | | | | | | | | | | | |
| | erm(41) T28C | | | | | | | | | | | 18 | 0 |
| | MB pos | | | | | | | | | | | | |
| | erm(41) T28C | | | | | | | | | | | 0 | 97 |
| | MB neg | | | | | | | | | | | | |

pos., positive; neg., negative.
bol = M. abscessus subsp. bolletii,
abs = M. abscessus subsp. abscessus,
mas = M. abscessus subsp. massiliense
*The 23S rrl MB was designed to detect wild-type of 2058-2059 alleles, and mutations in 2058 or 2059 will fail to show amplification curve. The negative amplification curve for 23S rrl MB is regarded as 23S rrl: 2058-2059 mutation result.

TABLE 6

| | | | | erm(41): | erm(41) | 23s rrl: |
|---|---|---|---|---|---|---|
| sample | abscessus | massiliense | bolletii | T28C | truncation | 2058-2059 |
| 55000 | abscessus | — | — | Wild-type | Wild-type | Wild-type |
| 55001 | abscessus | — | — | Wild-type | Wild-type | Wild-type |
| 55002 | abscessus | — | — | Wild-type | Wild-type | Wild-type |
| 55003 | abscessus | — | — | Wild-type | Wild-type | Wild-type |
| 55004 | abscessus | — | — | Wild-type | Wild-type | Wild-type |
| 55005 | abscessus | — | — | Wild-type | Wild-type | Wild-type |
| 55006 | abscessus | — | — | Wild-type | Wild-type | Wild-type |
| 55007 | abscessus | — | — | Wild-type | Wild-type | Wild-type |
| 55008 | abscessus | — | — | Wild-type | Wild-type | Wild-type |
| 55009 | abscessus | — | — | Wild-type | Wild-type | Wild-type |
| 55010 | abscessus | — | — | Wild-type | Wild-type | Wild-type |
| 55011 | abscessus | — | — | Wild-type | Wild-type | Wild-type |
| 55012 | abscessus | — | — | Wild-type | Wild-type | Wild-type |
| 55013 | abscessus | — | — | Wild-type | Wild-type | Wild-type |
| 55014 | abscessus | — | — | Wild-type | Wild-type | Wild-type |
| 55015 | abscessus | — | — | Wild-type | Wild-type | Wild-type |
| 55016 | abscessus | — | — | T28C | Wild-type | Wild-type |
| 55017 | abscessus | — | — | T28C | Wild-type | Wild-type |
| 55018 | abscessus | — | — | T28C | Wild-type | Wild-type |
| 55019 | abscessus | — | — | Wild-type | Wild-type | Wild-type |
| 55020 | abscessus | — | — | T28C | Wild-type | Wild-type |
| 55021 | abscessus | — | — | T28C | Wild-type | Wild-type |
| 55022 | abscessus | — | — | Wild-type | Wild-type | Wild-type |
| 55023 | abscessus | — | — | Wild-type | Wild-type | Wild-type |
| 55024 | abscessus | — | — | Wild-type | Wild-type | Wild-type |
| 55025 | abscessus | — | — | Wild-type | Wild-type | Wild-type |
| 55026 | abscessus | — | — | Wild-type | Wild-type | Wild-type |
| 55027 | abscessus | — | — | Wild-type | Wild-type | Wild-type |
| 55028 | abscessus | — | — | Wild-type | Wild-type | Wild-type |
| 55029 | abscessus | — | — | Wild-type | Wild-type | Wild-type |
| 55030 | abscessus | — | — | Wild-type | Wild-type | Wild-type |
| 55031 | abscessus | — | — | Wild-type | Wild-type | Wild-type |
| 55032 | abscessus | — | — | Wild-type | Wild-type | Wild-type |
| 55033 | abscessus | — | — | Wild-type | Wild-type | Wild-type |
| 55034 | abscessus | — | — | Wild-type | Wild-type | Wild-type |
| 55035 | abscessus | — | — | Wild-type | Wild-type | Wild-type |
| 55036 | abscessus | — | — | Wild-type | Wild-type | Wild-type |
| 55037 | abscessus | — | — | Wild-type | Wild-type | Wild-type |
| 55038 | abscessus | — | — | Wild-type | Wild-type | Wild-type |
| 55039 | abscessus | — | — | Wild-type | Wild-type | Wild-type |
| 55040 | abscessus | — | — | Wild-type | Wild-type | Wild-type |
| 55041 | abscessus | — | — | Wild-type | Wild-type | Wild-type |
| 55043 | abscessus | — | — | T28C | Wild-type | Wild-type |
| 55044 | abscessus | — | — | T28C | Wild-type | Wild-type |
| 55045 | abscessus | — | — | T28C | Wild-type | Wild-type |
| 55046 | abscessus | — | — | T28C | Wild-type | Wild-type |
| 55047 | abscessus | — | — | T28C | Wild-type | Wild-type |
| 55048 | abscessus | — | — | T28C | Wild-type | Wild-type |
| 55049 | abscessus | — | — | T28C | Wild-type | Wild-type |
| 55050 | abscessus | — | — | T28C | Wild-type | Wild-type |
| 55051 | abscessus | — | — | Wild-type | Wild-type | Wild-type |
| 55052 | abscessus | — | — | Wild-type | Wild-type | Wild-type |
| 55053 | abscessus | — | — | Wild-type | Wild-type | Wild-type |
| 55054 | abscessus | — | — | Wild-type | Wild-type | Wild-type |
| 55055 | abscessus | — | — | Wild-type | Wild-type | Wild-type |
| 55056 | abscessus | — | — | T28C | Wild-type | Wild-type |
| 55057 | abscessus | — | — | Wild-type | Wild-type | Wild-type |
| 55058 | abscessus | — | — | Wild-type | Wild-type | Wild-type |
| 55059 | — | — | bolletii | Wild-type | Wild-type | Wild-type |
| 55060 | — | — | bolletii | Wild-type | Wild-type | Wild-type |
| 55061 | — | — | bolletii | Wild-type | Wild-type | Wild-type |
| 55062 | — | — | bolletii | Wild-type | Wild-type | Wild-type |
| 55063 | — | — | bolletii | Wild-type | Wild-type | Wild-type |
| 55064 | — | — | bolletii | Wild-type | Wild-type | Wild-type |
| 55065 | — | — | bolletii | Wild-type | Wild-type | Wild-type |
| 55066 | — | massiliense | — | Wild-type | truncation | Wild-type |
| 55067 | — | massiliense | — | Wild-type | truncation | Wild-type |
| 55068 | — | massiliense | — | Wild-type | truncation | Mutant |
| 55069 | — | massiliense | — | Wild-type | truncation | Wild-type |
| 55070 | — | massiliense | — | Wild-type | truncation | Wild-type |
| 55071 | — | massiliense | — | Wild-type | truncation | Wild-type |
| 55072 | — | massiliense | — | Wild-type | truncation | Wild-type |
| 55073 | — | massiliense | — | Wild-type | truncation | Wild-type |
| 55074 | — | massiliense | — | Wild-type | truncation | Wild-type |
| 55075 | — | massiliense | — | Wild-type | truncation | Wild-type |
| 55076 | — | massiliense | — | Wild-type | truncation | Wild-type |
| 55077 | — | massiliense | — | Wild-type | truncation | Wild-type |
| 55078 | — | massiliense | — | Wild-type | truncation | Wild-type |
| 55079 | — | massiliense | — | Wild-type | truncation | Wild-type |
| 55080 | — | massiliense | — | Wild-type | truncation | Wild-type |
| 55081 | — | massiliense | — | Wild-type | truncation | Wild-type |
| 55082 | — | massiliense | — | Wild-type | truncation | Wild-type |
| 55083 | — | massiliense | — | Wild-type | truncation | Wild-type |
| 55084 | — | massiliense | — | Wild-type | truncation | Wild-type |
| 55085 | — | massiliense | — | Wild-type | truncation | Wild-type |
| 55086 | — | massiliense | — | Wild-type | truncation | Wild-type |
| 55087 | — | massiliense | — | Wild-type | truncation | Wild-type |
| 55088 | — | massiliense | — | Wild-type | truncation | Wild-type |
| 55089 | — | massiliense | — | Wild-type | truncation | Wild-type |
| 55090 | — | massiliense | — | Wild-type | truncation | Wild-type |
| 55091 | — | massiliense | — | Wild-type | truncation | Wild-type |
| 55092 | — | massiliense | — | Wild-type | truncation | Wild-type |
| 55093 | — | massiliense | — | Wild-type | truncation | Wild-type |
| 55094 | — | massiliense | — | Wild-type | truncation | Wild-type |
| 55095 | — | massiliense | — | Wild-type | truncation | Wild-type |
| 55096 | — | massiliense | — | Wild-type | Wild-type | Wild-type |
| 55097 | — | massiliense | — | Wild-type | truncation | Wild-type |
| 55099 | — | massiliense | — | Wild-type | truncation | Wild-type |
| 47350 | — | — | bolletii | Wild-type | Wild-type | Wild-type |
| 51401 | abscessus | — | — | Wild-type | Wild-type | Mutant |
| 51402 | abscessus | — | — | T28C | Wild-type | Wild-type |
| 51412 | abscessus | — | — | Wild-type | Wild-type | Mutant |
| 51712 | abscessus | — | — | T28C | Wild-type | Wild-type |
| 51722 | — | massiliense | — | Wild-type | truncation | Wild-type |
| 51749 | — | — | bolletii | Wild-type | Wild-type | Wild-type |
| 51775 | abscessus | — | — | Wild-type | Wild-type | Wild-type |
| 51396 | abscessus | — | — | T28C | Wild-type | Wild-type |
| 51404 | — | massiliense | — | Wild-type | truncation | Wild-type |
| 51414 | abscessus | — | — | Wild-type | Wild-type | Wild-type |
| 51420 | abscessus | — | — | Wild-type | Wild-type | Wild-type |
| 51421 | abscessus | — | — | Wild-type | Wild-type | Mutant |
| 51425 | — | — | bolletii | Wild-type | Wild-type | Wild-type |
| 51764 | — | massiliense | — | Wild-type | truncation | Wild-type |
| 51766 | abscessus | — | — | T28C | Wild-type | Wild-type |
| 51778 | abscessus | — | — | Wild-type | Wild-type | Wild-type |

1. A method for detecting different species of the Mycobacterium abscessus and detecting a mutation, comprising:
preparing an assay;
including a molecular beacon probe in the assay to determine a species of the Mycobacterium abscessus;
including a primer in the assay to detect a mutation;
performing a real time-polymerase chain reaction (RT-PCR) on the assay, and
analyzing the assay for the species of the Mycobacterium abscessus and the mutation.

2. The method of claim 1, wherein the assay is a two-tube assay.

3. The method of claim 1, wherein the assay is an eight-tube assay.

4. The method of claim 1, where the molecular beacon probe comprise SEQ ID NOS. 3, 6 or 7, or a combination thereof.

5. An assay for detecting different species of the Mycobacterium abscessus and detecting a mutation comprising:
a forward primer for MAB2248 represented by SEQ ID NO. 1;
a reverse primer for MAB2248 represented by SEQ ID NO. 2;
a molecular beacon probe for detecting M. abscessus subsp. bolletii reperesented by SEQ ID NO. 3;
a forward primer for MAB2830 represented by SEQ ID NO. 4;
a reverse primer for MAB2830 represented by SEQ ID NO. 5;
a molecular beacon probe for detecting M. abscessus subsp. abscessus represented by SEQ ID NO. 6;
a molecular beacon probe for detecting M. abscessus subsp. massiliense represented by SEQ ID NO. 7;
a forward primer for 23S rrl represented by SEQ ID NO. 8;
a reverse primer for 23S rrl represented by SEQ ID NO. 9;
a molecular beacon probe for detecting the wild type of 23S rrl represented by SEQ ID NO. 10;
a forward primer for erm(41) A represented by SEQ ID NO. 11;
a reverse primer for erm(41) A represented by SEQ ID NO. 12;
a molecular beacon probe for erm(41) A represented by SEQ ID NO. 13;
a forward primer for erm(41) T28C represented by SEQ ID NO. 14;
a reverse primer for erm(41) T28C represented by SEQ ID NO. 15; and
a molecular beacon probe for detecting erm(41) T28C represented by SEQ ID NO. 16.
